(19) United States
(12) Patent Application Publication
(10) Pub. No.: US 2018/0055978 A1
(43) Pub. Date: Mar. 1, 2018

(54) RESERVOIR AND FEEDING SYSTEM FOR USE WITH BREAST PUMPS

(71) Applicant: Katherine M Ong, Bethpage, NY (US)

(72) Inventor: Katherine M Ong, Bethpage, NY (US)

(21) Appl. No.: 15/245,617

(22) Filed: Aug. 24, 2016

(51) Int. Cl. A61M 1/06

(52) U.S. Cl. CPC A61M 1/068; A61M 1/066

(57) ABSTRACT

A breast milk storage and delivery system including: a breast attachment configured to receive a supply of breast milk; a reservoir operatively connected to the breast attachment interface, the reservoir having: an inlet end, the inlet end having an attachment mechanism configured to receive milk supplied to the reservoir from the breast attachment interface; a reservoir portion having an internal volume for storing a portion of pumped breast milk; and a delivery end, the delivery end including an outlet, and an outlet attachment mechanism; and a nipple, the nipple being operatively connected to the outlet attachment mechanism of the reservoir.

### BACKGROUND

The present invention relates to various systems and methods for collecting and delivering breast milk to infants.

In particular the present invention relates to breast pumps along with the reservoirs commonly used therewith. Typical breast pumps use a suction device or a vacuum generator which is used in conjunction with a breast interface so as to remove breast milk from a mother's breast and store the pumped breast milk into a reservoir. The pumped breast milk can then be transferred from the reservoir into a feeding bottle for delivery to an infant.

Using the systems and methods presently available can be exceedingly difficult for mothers having multiple breastfeeding children, i.e. mothers of twins, to meet the demands of each child simultaneously as it can be difficult to properly position and hold each child in an appropriate breastfeeding position. For mothers that prefer pumping to direct breast feeding such systems also require that the child wait to eat while the breast milk is collected as such systems only include a single aperture which can only be attached to one of a nipple attachment or the breast attachment.

### BRIEF DESCRIPTION OF THE INVENTION

Applicant has recognized that a need exists which would allow a mother of more than one child to simultaneously pump breast milk from both breasts, while simultaneously feeding more than one child. Another recognized need is to enable a mother to feed a single child while also simultaneously pumping and storing excess breast milk.

As such, the present invention overcomes many of the disadvantages of the various systems of the prior art by providing a breast pump and associated breast attachments being configured to attach to, and pump from, both breasts simultaneously.

In order to overcome many of these advantages various embodiments of a breast milk storage and delivery system are contemplated herein. The system can include a breast attachment interface configured to receive a supply of breast milk, for example from a breast pump or other vacuum generating device. The system can then include a reservoir being configured to be operatively connected to the breast attachment interface and thus store milk being pumped from a user's breast or breasts. This reservoir can include an inlet end, the inlet end having an attachment mechanism configured to receive milk supplied to the reservoir from the breast attachment interface; a reservoir portion which includes, or allows for, an internal volume for storing a portion of pumped breast milk; and a delivery end, the delivery end including an outlet, and an outlet attachment mechanism. The system can then be provided with a nipple, wherein the nipple can be configured to be operatively connected to the outlet attachment mechanism of the reservoir. In this manner breast milk can be received at a first end of the reservoir from the breast attachment, and then a baby can latch on to the nipple being provided at a second end of the reservoir in which case the user can simultaneously pump and feed a child using a breast pump device.

In some additional embodiments the reservoir portion can be formed of a thin collapsible sidewall. In some such embodiments the reservoir portion is formed of a rigid shape retaining sidewall. Alternatively, in some embodiments the reservoir can be formed of a semi-rigid material.

In some embodiments the reservoir can be connected at the inlet end to the breast attachment interface using a threaded connection, however various snap fit or press fit connections might also prove to be suitable.

In yet additional embodiments the reservoir can be provided with a slit valve, wherein the slit valve can be provided about the outlet of the reservoir.

In yet additional embodiments the system can be provided with a selectively openable and closeable valve provided between the nipple and the reservoir. In some such embodiments the nipple can be provided with a nipple interface, and wherein the reservoir outlet can be provided with a valve protrusion, wherein the valve protrusion is configured to nest within the nipple interface, and where the nipple interface is configured to slide axially with respect to valve protrusion. In some alternative embodiments of this feature the nipple interface can further include a radially inwardly protruding sealing flange, and wherein the valve protrusion further comprises a radially outwardly protruding sealing flange, wherein the radially outwardly protruding sealing flange and the radially inwardly protruding sealing flange are configured to engage one another in an extended configuration so as to limit the axial translation of the nipple with respect to the reservoir and prevent unwanted separation.

In yet additional embodiments the nipple can also include an exterior malleable portion and an internal connection interface portion, wherein the internal connection interface portion is formed of a material having a higher durometer than the exterior malleable portion. In some such embodiments the exterior malleable portion can be overmolded onto the internal connection interface portion.

Also contemplated herein is a method of collecting and feeding breastmilk, the method having various steps, including: attaching a breast attachment to a user's breast; attaching a reservoir having an inlet end and an outlet end to the breast attachment, the reservoir being configured to interface with the breast attachment at the an inlet end; attaching a malleable nipple about the outlet end of the reservoir; collecting a quantity of breast milk through the inlet end into the reservoir; and delivering a portion of the quantity of breast milk through the outlet and through the nipple to an alternative user.

In some embodiments this method can also include the step of selectively opening or closing a valve provided between the nipple and the reservoir.

In some embodiments this method can also include the step of removing the reservoir from the breast attachment and placing a cap about the inlet end.

In some embodiments this method can also include the step of removing the nipple from the reservoir and placing a cap about the outlet end.

In some embodiments this method can also include the step of removing the nipple from the reservoir and placing a cap about the outlet end.

In some embodiments this method can also include the steps of: attaching a second breast attachment to a user's second breast; attaching a second reservoir having an inlet end and an outlet end to the second breast attachment, the second reservoir being configured to interface with the second breast attachment at the an inlet end; attaching a second malleable nipple about the outlet end of the second reservoir; collecting a second quantity of breast milk through the inlet end into the second reservoir; and delivering a portion of the second quantity of breast milk through the outlet and through the second nipple to a second alternative user.

These aspects of the invention are not meant to be exclusive and other features, aspects, and advantages of the present invention will be readily apparent to those of ordinary skill in the art when read in conjunction with the following description, appended claims, and accompanying drawings. Further, it will be appreciated that any of the various features, structures, steps, or other aspects discussed herein are for purposes of illustration only, any of which can be applied in any combination with any such features as discussed in alternative embodiments, as appropriate.

### BRIEF DESCRIPTION OF THE DRAWINGS

FIG. 1 illustrates a perspective view of a user utilizing a reservoir and feeding system along with an associated breast pump in accordance with various aspects of the present invention;

FIG. 2 illustrates a perspective view of a user utilizing a reservoir and feeding system along with an associated breast pump in accordance with various aspects of the present invention in an alternative usage manner;

FIG. 3 illustrates a perspective view of a first embodiment of a reservoir and feeding system for use with various breast pumps in accordance with various aspects of the present invention;

FIG. 4 illustrates an exploded perspective view of the reservoir and feeding system of FIG. 3;

FIG. 5 illustrates an exploded side cross-sectional view of the reservoir and feeding system of FIG. 3;

FIGS. 6A-B illustrate front and rear perspective views of a nipple assembly for use with the reservoir and feeding system of FIG. 3;

FIG. 7 illustrates an exploded side cross-sectional view of the nipple assembly of FIGS. 6A-B;

FIG. 8 illustrates a perspective view of a reservoir for use with the reservoir and feeding system of FIG. 3

FIG. 9 illustrates an end view of the reservoir of FIG. 8;

FIG. 10 illustrates a perspective cross-sectional view of the reservoir of FIG. 8;

FIG. 11 illustrates a side view of a second embodiment of a reservoir and feeding system for use with various breast pumps in accordance with various aspects of the present invention;

FIG. 12 illustrates a side perspective view of the reservoir and feeding system of FIG. 11;

FIG. 13 illustrates a side exploded view of reservoir and feeding system of FIG. 11;

FIG. 14 illustrates a side exploded cross-sectional view of the reservoir and feeding system of FIG. 11;

FIG. 15 illustrates a side exploded view of a nipple assembly for use with the reservoir and feeding system of FIG. 11;

FIGS. 16A-B illustrate front and rear perspective exploded views of the nipple assembly of FIG. 15;

FIG. 17 illustrates a side cross-sectional view of the nipple assembly of FIG. 15;

FIG. 18 illustrates an end perspective view of a reservoir for use in the reservoir and feeding system of FIG. 11;

FIGS. 19A-B illustrate side cross sectional views of the reservoir of FIG. 18 in respective open and closed valve states;

FIGS. 20A-B illustrate side and cross-sectional views of the reservoir and feeding system of FIG. 11 in a closed valve state; and

FIGS. 21A-B illustrate side and cross-sectional views of the reservoir and feeding system of FIG. 11 in an open valve state.

### DETAILED DESCRIPTION

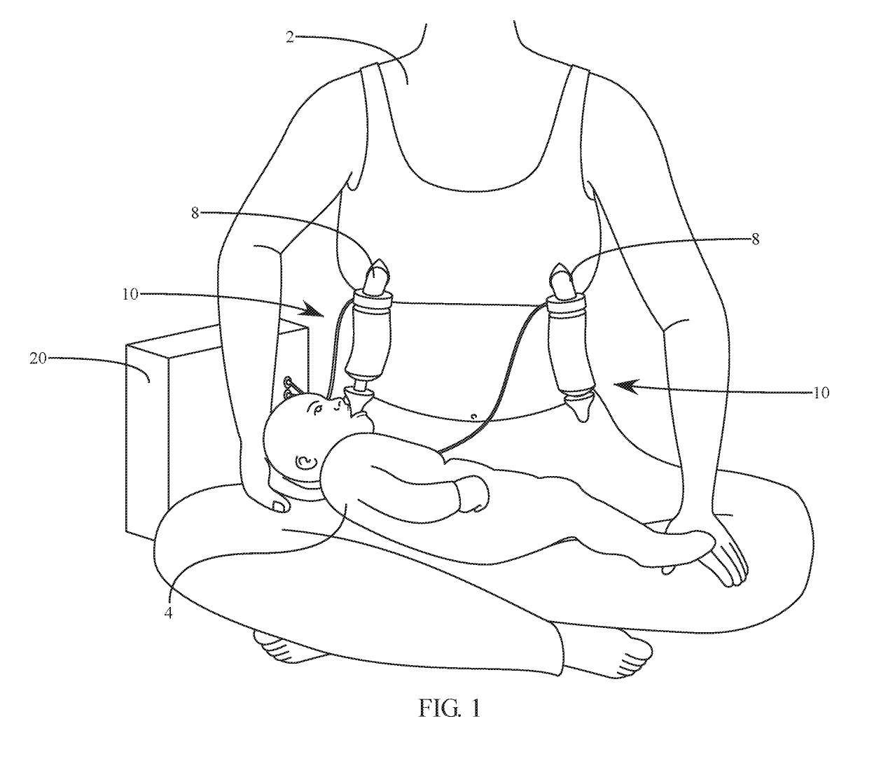

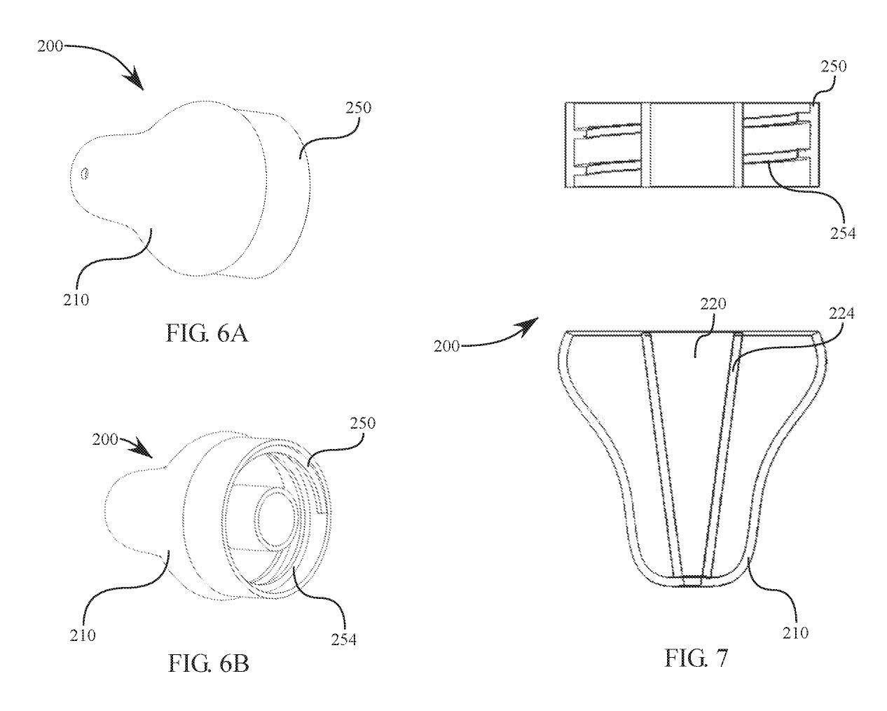

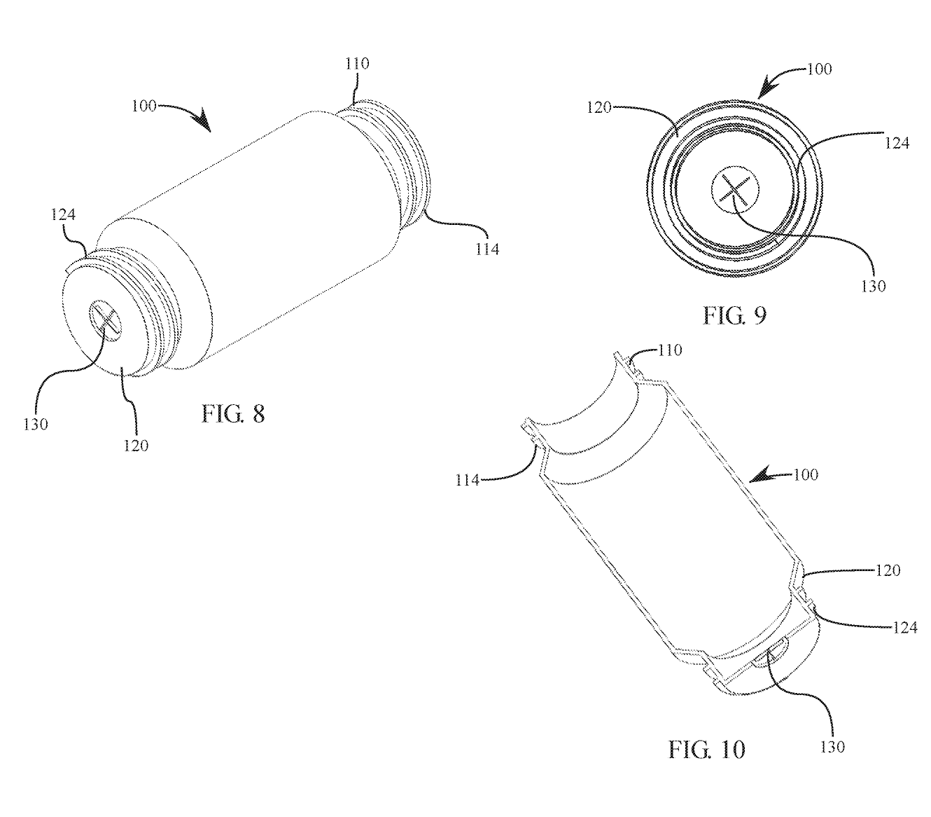

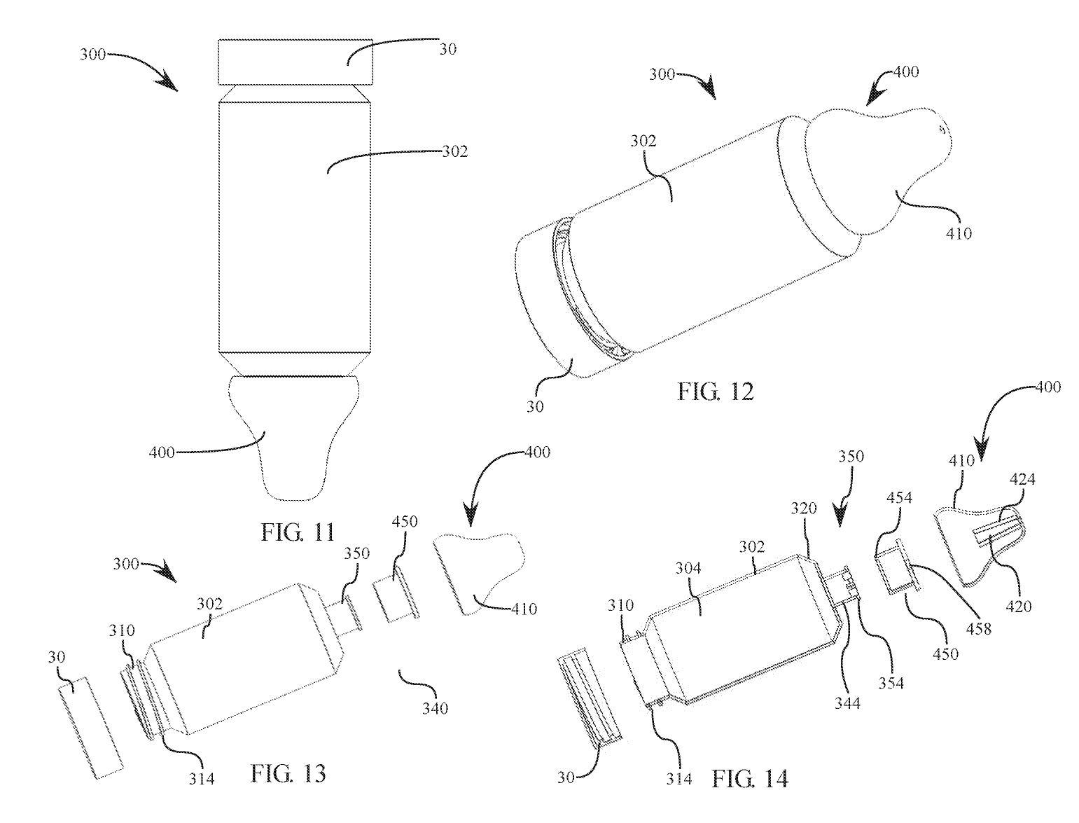

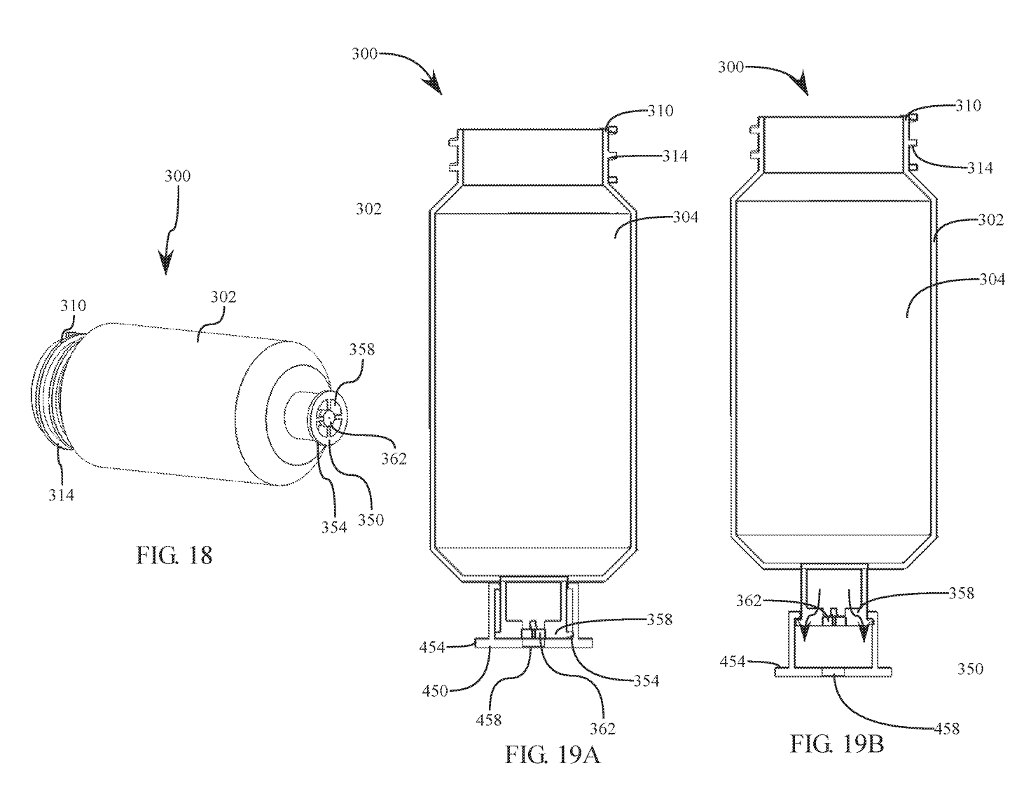

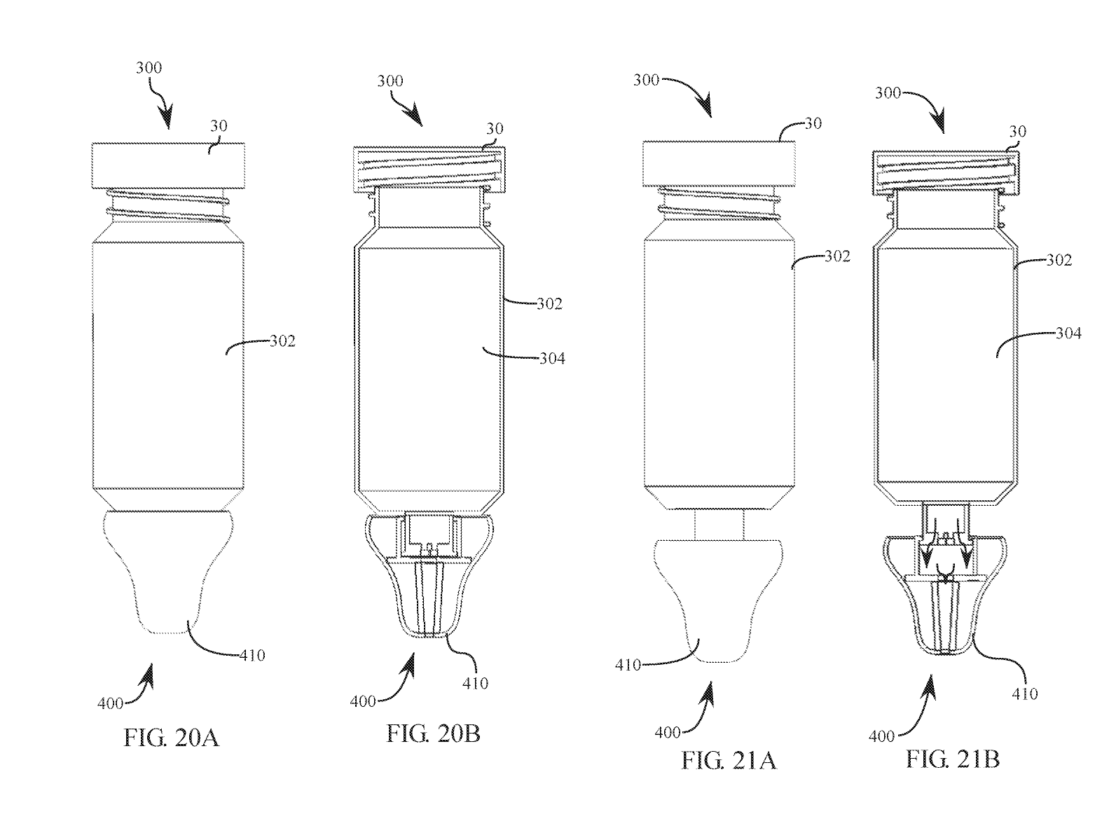

As discussed briefly above, and as shown herein in FIGS. 1-21, the present invention relates to a breast milk storage and delivery system 10. The breast milk storage and delivery system 10 is designed for use by mothers having one or more breastfeeding children in conjunction with a breast pump or vacuum generating device 20 which can, in conjunction with a breast attachment interface 8, be utilized to extract breast milk from the mother as well as simultaneously, or at a later time, deliver the breast milk to a child 4 or 6, or a plurality of children while simultaneously pumping.

In the embodiments shown, the system can include a reservoir 100 which can be operatively connected to the breast attachment interface 8. In the embodiments shown the reservoir 100 can include an inlet end 110, the inlet end 110 having an attachment mechanism 124 being configured to receive breast milk from the breast attachment interface 8. It will be appreciated that the attachment mechanism 124 shown herein includes male and female threads. However, it will be appreciated that numerous alternative attachment means, such as snap or press fit would be suitable for attaching the reservoir to the breast attachment interface 8. Alternative structures can also include valves, flexible tubing connections, or any other well appreciated means of making a fluid connection. It has been recognized that in the particular embodiments shown that advantages are realized through having direct and rigid coupling so as to aid in positioning the reservoir during pumping and simultaneous delivery of the collected milk.

It will be appreciated that the reservoir can have an increased internal volume forming a reservoir portion 102 having sufficient volume to contain all of the breast milk which might potentially be collected from a single breast, and can also be enough to contain the milk of two complete breasts.

The reservoir 100 can also include a valve 130 provided at an outlet end 120. The outlet end 120 can further include an outlet attachment mechanism 124 configured to attach to a nipple 200 having an interior channel 220 providing fluid communication to a tip portion. In this manner, a child 4 or 6 can latch onto and receive breast milk from a second outlet end of the reservoir 100 using the nipple 200, while not interfering with the vacuum generator 20 and the pumping of milk into the reservoir 100.

It has been recognized that many women prefer to use a breast pump over direct breast feeding due to the reliability of extraction as well as the increased comfort of the breast attachment interfaces over direct breast feeding. Additionally it has been recognized that for certain mothers and children the present invention can overcome difficulties in feeding children that have trouble latching, or for other reasons it has become advantageous to pump breast milk rather than direct breastfeed.

The valve 130 can be provided as any number of suitable valves, however, particular advantages have been achieved through the use of a slit valve. The slit valve typically involves a highly resilient membrane type material, such as silicone or rubber, into which a pair of slits can be cut which seal to themselves under zero or low pressure situations, but which open under pressure to allow fluid to pass therethrough. Such a valve eliminates any leaking through the nipple 200 when a child is not latched thereon and actively sucking on the nipple 200.

Certain advantages have also been realized in the system 10 through the use of a reservoir 100 which is formed of a flex form or shape retaining material. In this manner the reservoir can be deformed and bent so as to provide a proper approach angle of the nipple 200 to the mouth of the breastfeeding child 4 in a manner such that the preferred shape is retained by the material once achieved. The deformable and/or flex form material allows one or even two children to be positioned in virtually any desired orientation or position and the reservoir to be deformed so as to properly allow the child to latch onto the nipple, thus making it far easier to simultaneously feed two children, while also simultaneously pumping breast milk into the reservoirs 100.

Additional advantages have been realized through the use of a thin and collapsible sidewall which allows for easy positioning of the bottle during use as well as easy storage and cleaning after use. However, in yet additional embodiments, the reservoir can be formed of rigid material which might be easier to sanitize and stand up to increased use or typical abuse associated therewith.

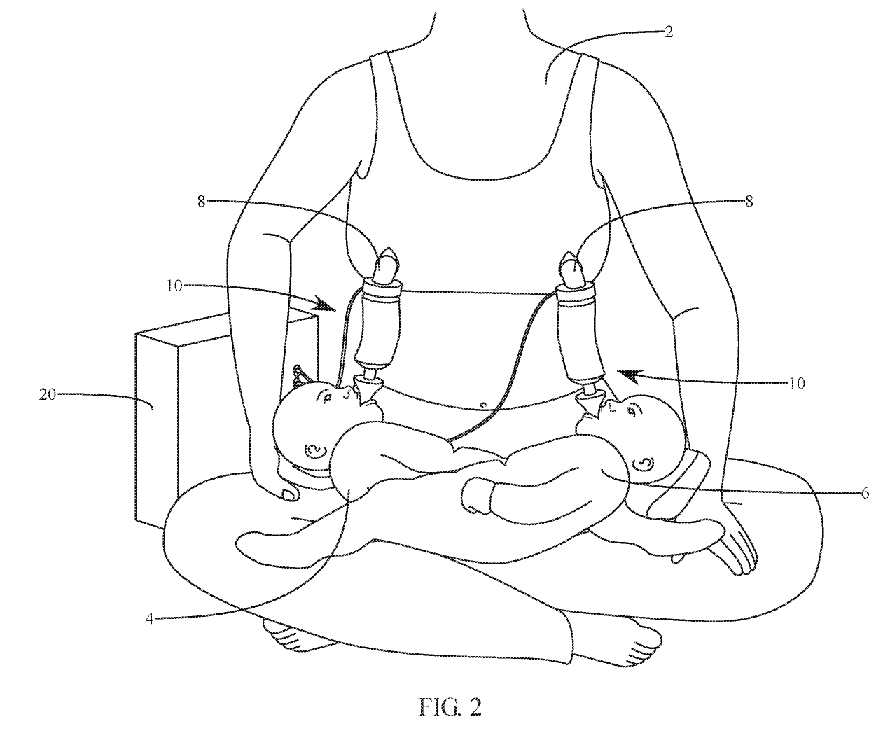

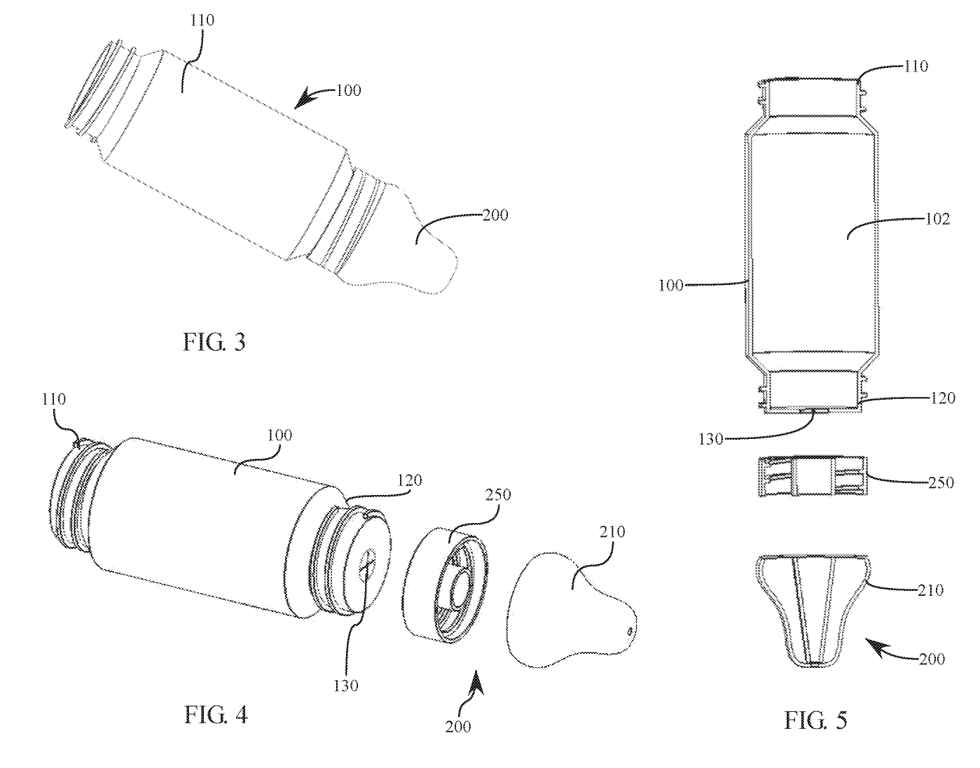

In the embodiments shown in FIGS. 3-10, the outlet end 120 of the reservoir can also include an attachment mechanism, such as the threads 124 shown. These threads can interface with corresponding threads 254 provided on an interior of the nipple 200.

In this embodiment, the nipple can be formed of a plurality of components including an exterior malleable portion 210 and an internal connection interface portion 250. In such an instance the exterior malleable portion 210 can be formed of a supple and resilient material which can be bonded, or overmolded onto, the internal connection interface portion 210 which includes the corresponding threads 254 or alternative connection means as recognized by those having skill in the art.

Yet additional advantages have been recognized through the use of a varying durometer between the exterior portion of the nipple and an interior channel sidewall 224. In some embodiments the exterior portion of the nipple can also have a varying durometer or hardness between a base portion and the tip portion so as to increase the bonding strength or increase the natural feel thus increasing the child's propensity of latching onto the nipple.

FIGS. 11-21 illustrate an alternative embodiment of a breast milk storage and delivery system 300. Similar to the embodiment discussed above, this alternative system 300 can include a reservoir 302 which can be operatively connected to the breast attachment interface 8. In the embodiments shown the reservoir 302 can include an inlet end 310, the inlet end 310 also having an attachment mechanism 314 being configured to receive breast milk from the breast attachment interface 8.

It will be appreciated that the reservoir can have an increased internal volume forming a reservoir portion 304 having sufficient volume to contain all of the breast milk which might potentially be collected from a single breast, and can also be enough to contain the milk of two complete breasts.

The reservoir 302 of this embodiment involves an alternative valve assembly 340 which can be selectively opened or closed. The valve assembly 340 can include an outlet interface 350 provided about an outlet end 320 and a nipple interface 450 provided on an interior portion of a nipple assembly 400.

It will be appreciated that outlet interface 350 can include a plurality of channels 358 being disposed about the exterior of a central post 362 provided about a center of the outlet interface. An aperture 458 corresponding in size to the central post 262 can then be provided to the nipple interface 450. It will then be appreciated that the nipple interface 450 can be allowed a small degree of axial motion with respect to the outlet interface wherein a compressed or closed state will cause the central post 362 to abut against and over the aperture 458 so as to block fluid communication between the interior channel 420 of the nipple and the interior of the reservoir 302. Further, in the expanded or open state the central post 362 to will be separated from the aperture 458 so as to allow fluid communication through the aperture 458 and thus allow milk delivery from the interior of the reservoir 302 through the aperture 458 and into the interior channel 420 of the nipple 410. This extended or open state is illustrated in FIGS. 19B, 21A, and 21B. In this manner, a child 4 or 6 can latch onto and receive breast milk from a second outlet end of the reservoir 302 using the nipple 410, while not interfering with the vacuum generator 20 and the pumping of milk into the reservoir 302.

It has been further recognized that the outlet interface 350 can be provided with a valve protrusion 344 having sufficient length so as to allow for proper closing and opening of the valve assembly 340 utilizing axial translation of the respective components. In order to reduce the chance of inadvertent separation of the nipple assembly 400 from the outlet interface 350 the valve protrusion 344 can be provided with a sealing flange 354 about its distal end which protrudes radially outward from the valve protrusion 344. The sealing flange can then interface with a corresponding sealing flange 454 feature provided on the interior of the nipple interface 450 which can protrude inwardly and thus limit separation of the nipple interface 450 from the outlet interface 350. The outlet interface 350 can thus be configured to nest into nipple interface 450 and can be of appropriate dimensions so as to provide a water tight seal and press fit onto one another.

It will be appreciated that one of ordinary skill would also be capable of reversing the sealing flange orientations and reversing the appropriate components such that the nipple interface 450 nested into the outlet interface 350. The necessary modifications also falling within the scope of this disclosure.

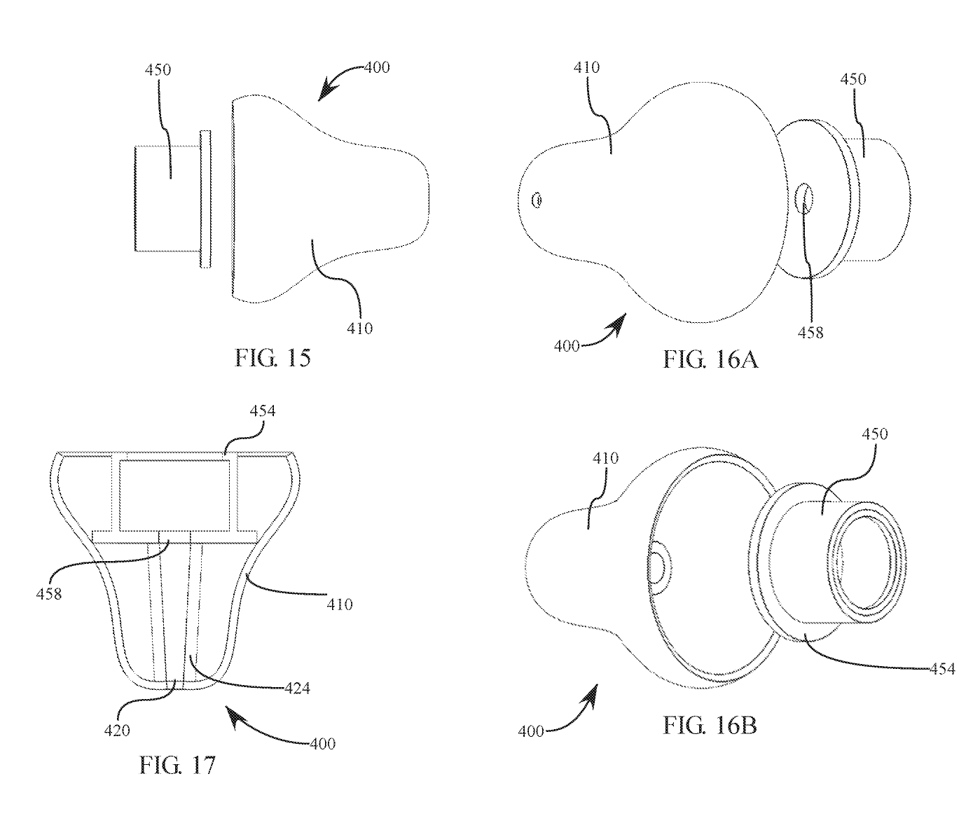

As shown in FIGS. 15-17 the nipple assembly 400 can also be provided as separate and distinct components, wherein the nipple interface 450 is formed of a separate and distinct material from the nipple portion 410. It will be appreciated that the nipple portion 410 can have a lower durometer, or be softer, as compared to the nipple interface 450. In this manner the nipple portion 410 can be overmolded onto or bonded to the nipple interface 450. The aperture 458 provided in the nipple interface 450 can provide fluid communication into the central channel 420 that allows for fluid communication to the tip of the nipple portion 410. In this manner the channel sidewall 424 can be sealed about a perimeter of the aperture 458 so as to prevent leaking.

The softer nipple portion can have varying durometers with respect to the base portion versus the tip portion, as discussed in relation to the previous embodiment. However the nipple interface 450 can be provided of a relatively rigid material which provides for a secure connection to the outlet interface 350.

It will also be appreciated that a cap 30 can be provided, and the valve closed so as to allow for storage of unused breast milk until a later time. The cap 30 can be provided about one or both ends of any of the reservoirs discussed above as appropriate so as to seal the reservoirs.

Also contemplated herein is a method of collecting and feeding breastmilk utilizing any one of the embodiments discussed above. The method can include the steps of attaching a breast attachment to a user's breast; attaching a reservoir having an inlet end and an outlet end to the breast attachment, the reservoir being configured to interface with the breast attachment at the an inlet end; attaching a malleable nipple about the outlet end of the reservoir; collecting a quantity of breast milk through the inlet end into the reservoir; and delivering a portion of the quantity of breast milk through the outlet and through the nipple to an alternative user.

This method can also include the steps of opening or closing a valve provided between the nipple and the reservoir so as to either seal the reservoir or open the fluid communication to the nipple and allow for feeding of the child.

When the child is done feeding the method of collecting and feeding breastmilk can then also include the step of removing the reservoir from the breast attachment and placing a cap about the inlet end as well as removing the nipple from the reservoir and placing a cap about the outlet end.

The method also contemplates the simultaneous pumping of and feeding of a child from each breast, wherein such a method would include the steps of: attaching a second breast attachment to a user's second breast; attaching a second reservoir having an inlet end and an outlet end to the second breast attachment, the second reservoir being configured to interface with the second breast attachment at the an inlet end; attaching a second malleable nipple about the outlet end of the second reservoir; collecting a second quantity of breast milk through the inlet end into the second reservoir; and delivering a portion of the second quantity of breast milk through the outlet and through the second nipple to a second alternative user.

The above description is merely illustrative. Having thus described several aspects of at least one embodiment of this invention including the preferred embodiments, it is to be appreciated that various alterations, modifications, and improvements will readily occur to those skilled in the art. Such alterations, modifications, and improvements are intended to be part of this disclosure, and are intended to be within the spirit and scope of the invention. Accordingly, the foregoing description and drawing are by way of example only. In particular certain features and structures are discussed with respect to one or more embodiments, it will also be appreciated that any features or structures discussed with respect to any particular embodiment can also be applied to any other particular embodiment without departing from the scope of this disclosure.

1. A breast milk storage and delivery system, the system comprising:
a breast attachment interface configured to receive a supply of breast milk;
a reservoir operatively connected to the breast attachment interface, the reservoir further comprising:
an inlet end, the inlet end having an attachment mechanism configured to receive milk supplied to the reservoir from the breast attachment interface;
a reservoir portion having an internal volume for storing a portion of pumped breast milk;
a delivery end, the delivery end including an outlet, and an outlet attachment mechanism; and
a nipple, the nipple being operatively connected to the outlet attachment mechanism of the reservoir.

2. The breast milk storage and delivery system of claim 1, wherein the reservoir portion is formed of a thin collapsible sidewall.

3. The breast milk storage and delivery system of claim 1, wherein the reservoir portion is formed of a rigid shape retaining sidewall.

4. The breast milk storage and delivery system of claim 1, wherein the reservoir is connected at the inlet end to the breast attachment interface using a threaded connection.

5. The breast milk storage and delivery system of claim 1, further comprising a slit valve provided about the outlet of the reservoir.

6. The breast milk storage and delivery system of claim 1, further comprising a selectively openable and closeable valve provided between the nipple and the reservoir outlet.

7. The breast milk storage and delivery system of claim 6, wherein the nipple includes a nipple interface, and wherein the reservoir outlet includes a valve protrusion, wherein the valve protrusion is configured to nest within the nipple interface, and where the nipple interface is configured to slide axially with respect to valve protrusion.

8. The breast milk storage and delivery system of claim 7, wherein the nipple interface further comprises a radially inwardly protruding sealing flange, and wherein the valve protrusion further comprises a radially outwardly protruding sealing flange, wherein the radially outwardly protruding sealing flange and the radially inwardly protruding sealing flange are configured to engage one another in an extended configuration so as to limit the axial translation of the nipple with respect to the reservoir and prevent unwanted separation.

9. The breast milk storage and delivery system of claim 6, wherein the selectively openable and closeable valve is opened by translating the nipple axially away from the reservoir.

10. The breast milk storage and delivery system of claim 1, wherein the nipple further comprises an exterior malleable portion and an internal connection interface portion, wherein the internal connection interface portion is formed of a material having a higher durometer than the exterior malleable portion.

11. The breast milk storage and delivery system of claim 10, wherein the exterior malleable portion is overmolded onto the internal connection interface portion.

12. A method of collecting and feeding breastmilk, the method comprising:
attaching a breast attachment to a user's breast;
attaching a reservoir having an inlet end and an outlet end to the breast attachment, the reservoir being configured to interface with the breast attachment at the inlet end;
attaching a malleable nipple about the outlet end of the reservoir;
collecting a quantity of breast milk through the inlet end into the reservoir; and
delivering a portion of the quantity of breast milk through the outlet and through the nipple to an alternative user.

13. The method of collecting and feeding breastmilk of claim 12, wherein the reservoir further comprises a thin collapsible sidewall.

14. The method of collecting and feeding breastmilk of claim 12, wherein the reservoir further comprising a slit valve provided about the outlet of the reservoir.

15. The method of collecting and feeding breastmilk of claim 12, further comprising the step of selectively opening or closing a valve provided between the nipple and the reservoir.

16. The method of collecting and feeding breastmilk of claim 12, further comprising the step of removing the reservoir from the breast attachment and placing a cap about the inlet end.

17. The method of collecting and feeding breastmilk of claim 12, further comprising the step of removing the nipple from the reservoir and placing a cap about the outlet end.

18. The method of collecting and feeding breastmilk of claim 17, further comprising the step of removing the nipple from the reservoir and placing a cap about the outlet end.

19. The method of collecting and feeding breastmilk of claim 12,
attaching a second breast attachment to a user's second breast;
attaching a second reservoir having an inlet end and an outlet end to the second breast attachment, the second reservoir being configured to interface with the second breast attachment at the an inlet end;
attaching a second malleable nipple about the outlet end of the second reservoir;
collecting a second quantity of breast milk through the inlet end into the second reservoir; and
delivering a portion of the second quantity of breast milk through the outlet and through the second nipple to a second alternative user.

20. A breast milk storage and delivery system, the system comprising:
a breast attachment configured to receive a supply of breast milk;
a reservoir operatively connected to the breast attachment interface, the reservoir further comprising:
an inlet end, the inlet end having an attachment mechanism configured to receive milk supplied to the reservoir from the breast attachment interface;
a reservoir portion being formed of a thin collapsible sidewall, the reservoir portion having an internal volume for storing a portion of pumped breast milk;
a delivery end, the delivery end including an outlet, and an outlet attachment mechanism; and
a nipple, the nipple being operatively connected to the outlet attachment mechanism of the reservoir, wherein the nipple further comprises an exterior malleable portion and an internal connection interface portion, wherein the internal connection interface portion is formed of a material having a higher durometer than the exterior malleable portion; and
a valve being provided between the nipple and the reservoir.
